(19) United States
(12) Patent Application Publication
(10) Pub. No.: US 2017/0231658 A1
(43) Pub. Date: Aug. 17, 2017

(54) ADAPTER FOR AN EXCISING APPARATUS

(71) Applicants: Jonathan Keith Jay, Naples, FL (US); Stephen Henry Miller, Naples, FL (US)

(72) Inventors: Jonathan Keith Jay, Naples, FL (US); Stephen Henry Miller, Naples, FL (US)

(21) Appl. No.: 15/433,087

(22) Filed: Feb. 15, 2017

Related U.S. Application Data
(60) Provisional application No. 62/295,594, filed on Feb. 16, 2016.

(51) Int. Cl. A61B 17/34; A61B 10/02

(52) U.S. Cl. CPC A61B 17/3423; A61B 10/0275; A61B 2017/3445; A61B 2017/3454

(57) ABSTRACT

An adapter (20) comprising a pair of rails (46) extending upwardly from a lower surface (38) and longitudinally in a direction from a long end (30) toward a short end (32) and converging to be parallel with the pair of edges (34) to define a third opening between the pair of rails (46) above the lower surface (38) for receiving the cannula (22) and stylet (24) transversely to the lower surface (38). An awning (48) positioned longitudinally between the first end and the second end and extending between and connecting the pair of rails (46) to define a first opening (42). The lower surface (38) defining a recess (50) disposed adjacent the short end (32) and extending from a second opening (44) into the channel (40) for receiving the flange (28) of the trocar (26) and defining a groove (52) for guiding the cannula (22) and the stylet (24) into the first opening (42). An indicia (54) extends from an upper surface (36) next-adjacent to the short end (32) to identify the second end for receiving the trocar (26).

### CROSS-REFERENCE TO RELATED APPLICATION

This application claims priority to U.S. Provisional Patent Application No. 62/295,594 which was filed on Feb. 16, 2016.

### BACKGROUND OF THE INVENTION

1. Field of the Invention

The invention relates to an adapter for guiding a cannula and a stylet into a passageway of a trocar having a flange to obtain a biopsy.

2. Description of the Prior Art

Trocars are well known in the art for guiding a cannula and a stylet to a surgical site to obtain a biopsy. Typically, during a medical procedure, a user will position and maintain a first end of trocar adjacent the surgical site by hand and then insert the cannula and stylet into a second end of the trocar. During this medical procedure, it is difficult for the user to maintain the first end adjacent the surgical site and to insert the cannula and stylet into the second end of the trocar. If the user does not precisely align the cannula and stylet into the second end of the trocar the user risk puncturing himself or another with the cannula or stylet. Accordingly, an adapter is typically attached to the second end of a trocar to guide the cannula and the stylet into second end.

Such prior art adapters typically comprise a long end and a short end with the short end being spaced from the long end and a pair of edges that extends between and converges to connect to the long end and the short end. These prior art adapters also comprise an upper surface that extends between the ends and the pair of edges and a lower surface that extends between the ends and the pair of edges. These prior art adapters further comprise a channel that extends between the ends and has a first opening and a second opening defined by the short end for receiving a trocar. An example of such a prior art adapter is shown in U.S. Pat. No. 5,220,926 issued to Wattiez et al. on Jan. 2, 2001. A problem with these prior art adapters is that they cannot be attached to an end of a trocar having a flange. For example, these prior art adapters cannot be attached to the end of the trocar shown in U.S. Patent Application Publication No. 2016/0058430 published to Miller et al. on Mar. 3, 2016. Another problem with these prior art adapters is that they cannot receive the cannula and the stylet transversely. Although these prior art adapters provide for a larger opening for guiding the cannula and the stylet into the trocar, there is still the risk that the user may not precisely align the cannula and the stylet with the larger opening and stab himself or another as he tries to insert the cannula and stylet into the trocar. Accordingly, there is a need for an adapter that can receive the cannula and the stylet to avoid a stabbing injury.

### SUMMARY OF THE INVENTION

The subject invention provides for an adapter comprising a pair of rails that extend upward from the lower surface and longitudinally in a direction from the long end toward the short end to define a third opening between the rails above the lower surface whereby the cannula and the stylet may be received transversely to the lower surface and guided by the pair of rails into the first opening.

### ADVANTAGES OF THE INVENTION

The invention in its broadest aspect provides for an adapter that overcomes the need to precisely align the cannula and the stylet with an opening to avoid the risk of a stabbing injury to the user or another.

### BRIEF DESCRIPTION OF THE DRAWINGS

Other advantages of the present invention will be readily appreciated, as the same becomes better understood by reference to the following detailed description when considered in connection with the accompanying drawings wherein:

FIG. 1 is a perspective view of the adapter;

FIG. 2 is top view of the adapter;

FIG. 3 is a bottom view of the adapter;

FIG. 4 is a side view of the adapter;

FIG. 5 is a back view of the adapter;

FIG. 6 is a front view of the adapter;

FIG. 7 is a perspective view showing the adapter being attached to the trocar;

FIG. 8 is a perspective view showing the adapter attached to the trocar;

FIG. 9 is a perspective view showing the adapter attached to the trocar and the cannula and stylet being guided into the trocar by the adapter;

FIG. 10 is a side view showing the adapter attached to the trocar and the cannula and stylet extending outwardly from an end of the trocar.

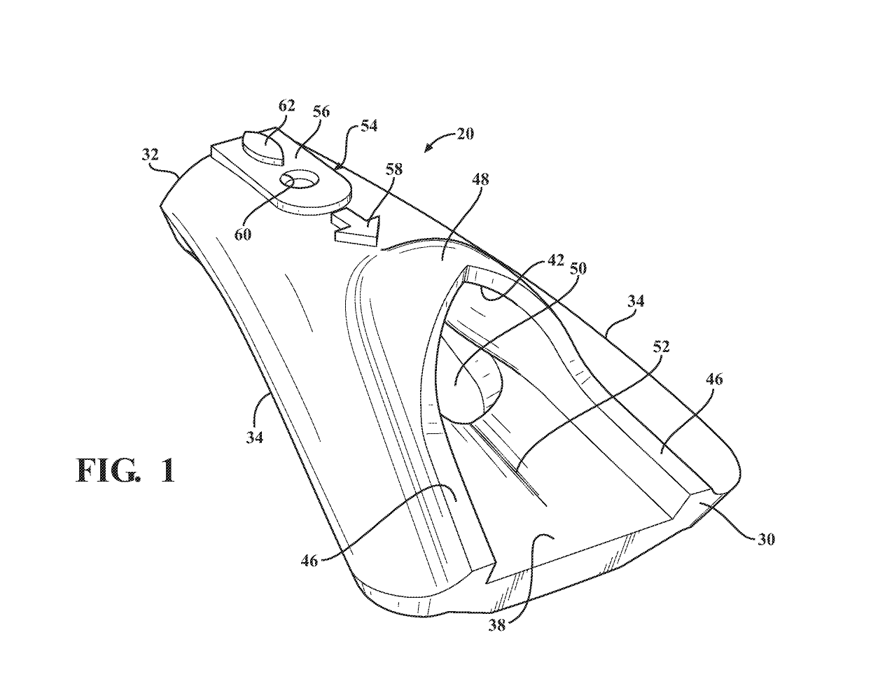

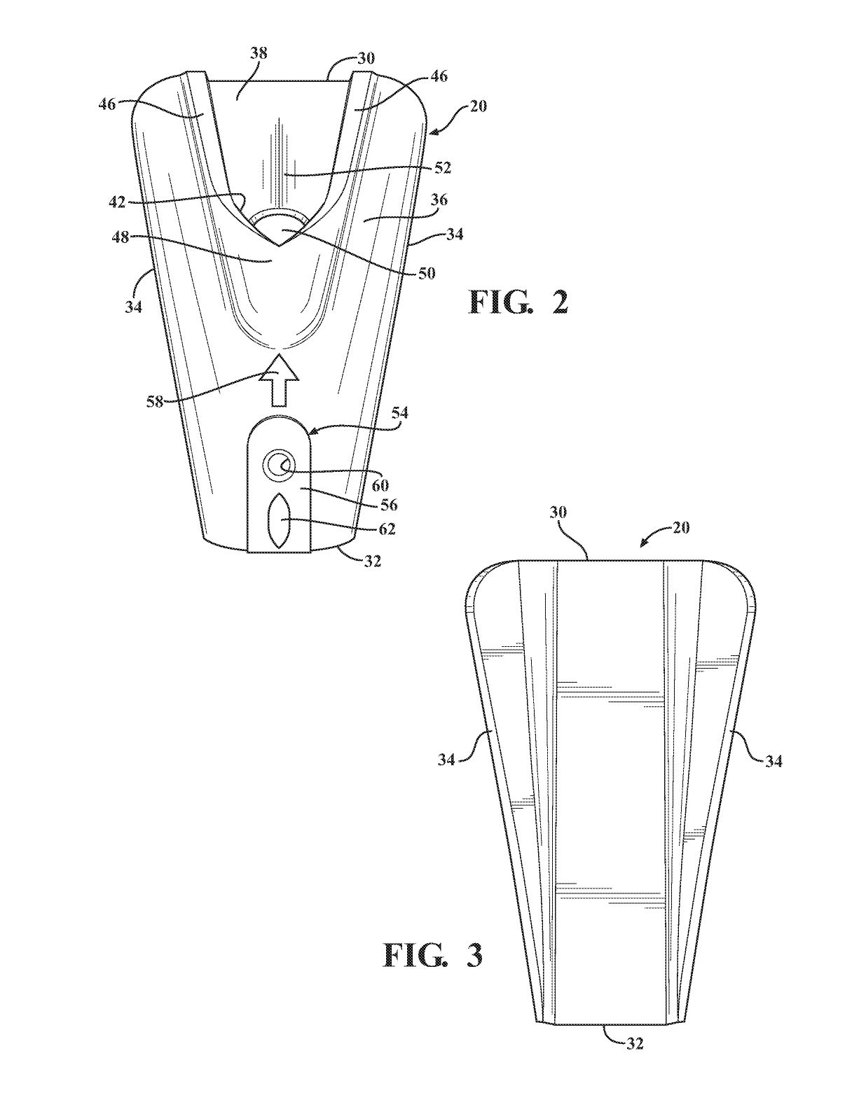

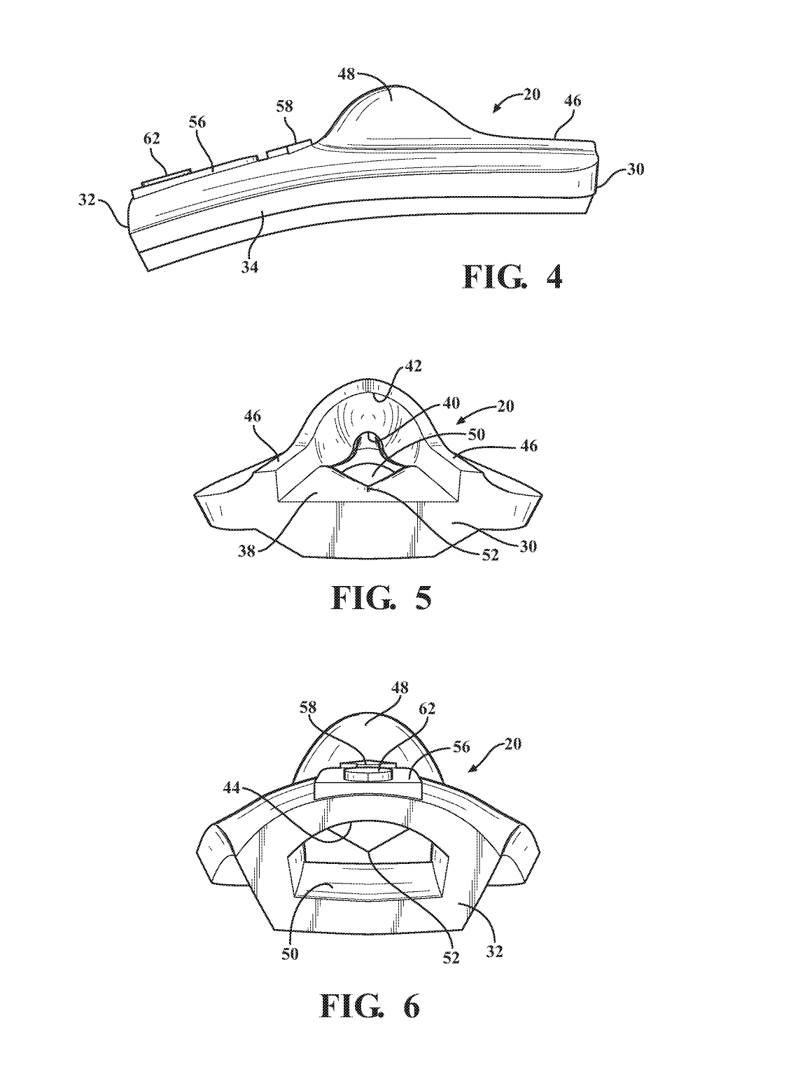

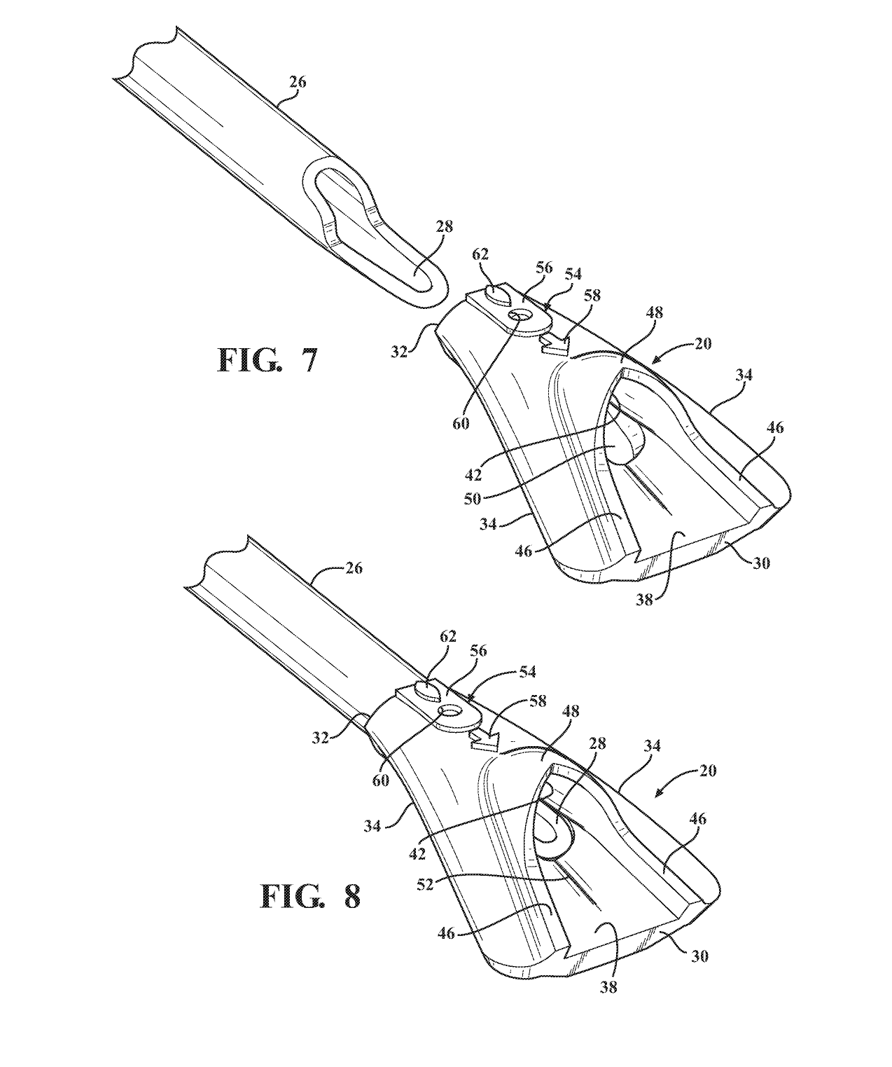

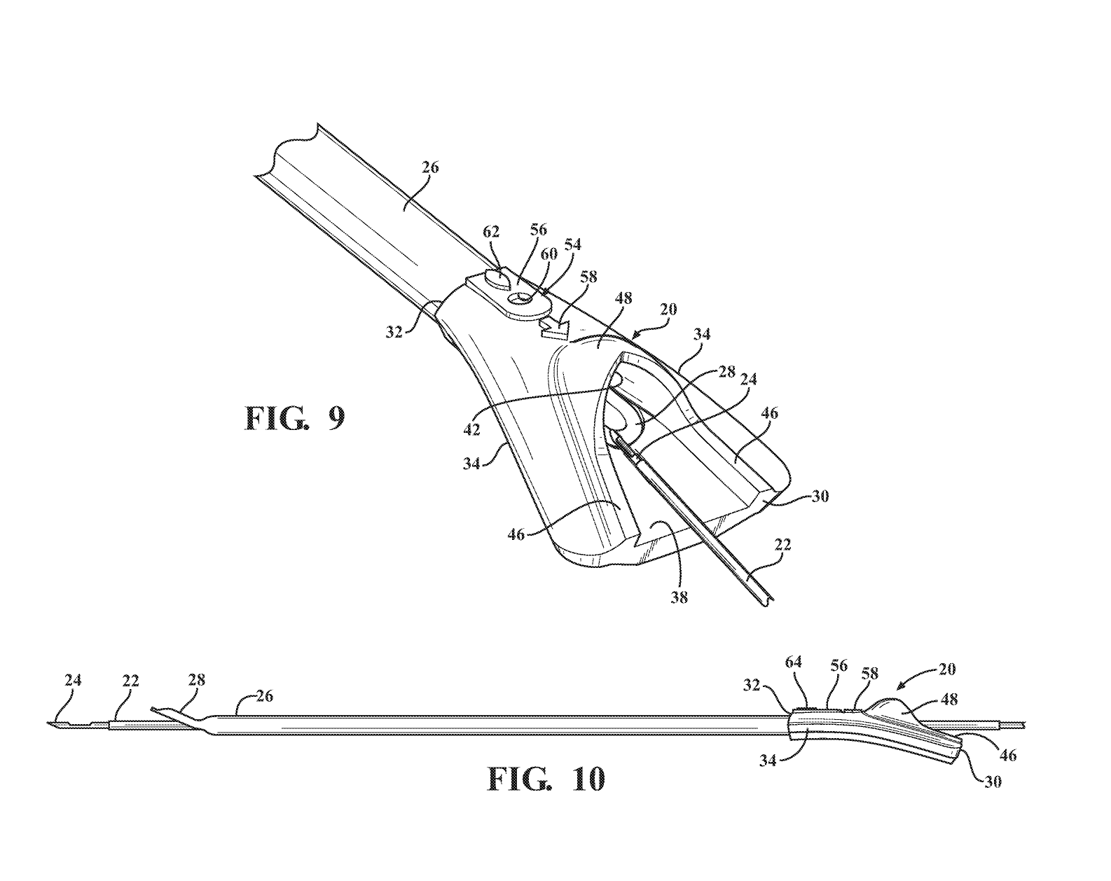

### DESCRIPTION OF THE ENABLING EMBODIMENT

Referring to the Figures, wherein like numerals indicate corresponding parts throughout the several views, an adapter 20 is generally shown for guiding a cannula 22 and stylet 24 into a passageway of a trocar 26 having a flange 28. The adapter 20 is of a trapezoidal shape and comprises a long end 30 and a short end 32 with the short end 32 being spaced from the long end 30. The adapter 20 also comprises a pair of edges 34 that are beveled inward and extend in an arcuate shape between and converge to connect to the long end 30 and the short end 32 to render the long end 30 longer in length than the short end 32 to define the trapezoidal shape. The adapter 20 has an upper surface 36 that extends between the ends 30, 32 and the pair of edges 34. The adapter 20 also has a lower surface 38 spaced from the upper surface 36 and extends between the ends 30, 32 and the pair of edges 34. The adapter 20 defines a channel 40 of a funnel shape and the channel 40 extends between the ends 30, 32. The adapter 20 also defines a first opening 42. The short end 32 defines a second opening 44 for receiving the trocar 26.

A pair of rails 46 extend upward from the lower surface 38 and longitudinally in a direction from the long end 30 toward the short end 32 and converge to be parallel with the pair of edges 34 to define a third opening between the pair of rails 46 and above the lower surface 38. The third opening allows for the adapter 20 to receive the cannula 22 and stylet 24 transversely to the lower surface 38. An awning 48 is positioned longitudinally between the first end and the second end and extends between and connects the pair of rails 46 to define the first opening 42. The lower surface 38 of the adapter 20 defines a recess 50 of an oval shape being disposed adjacent the short end 32 and extending from the second opening 44 into the channel 40 for receiving the flange 28 of the trocar 26. In an alternative embodiment, the recess 50 could be of a square, rectangular or alternative shape. It is to be appreciated that the shape and size of the recess 50 reflects the shape of the flange 28 of trocar 26, i.e., if the flange 28 of the trocar 26 is of a square shape than the shape of the recess 50 is of a square shape.

The lower surface 38 of the adapter 20 defines a groove 52 of a V-shape that is perpendicular to the long end 30 and extends from the first opening 42 to the recess 50. The groove 52 and the pair of rails 46 guide the cannula 22 and the stylet 24 into the first opening 42 when the stylet 24 and cannula 22 are positioned on the lower surface 38 and slid towards the first opening 42.

An indicia 54, generally indicated, extends from the upper surface 36 next-adjacent to the short end 32. The indicia 54 identifies the second end of the adapter 20 for receiving the trocar 26. The indicia 54 includes a first protrusion 56 of a rectangular shape that extends from the upper surface 36. The indicia 54 further includes a second protrusion 58 that extends from the upper surface 36 and has the shape of an arrow that points to the long end 30. The second protrusion 58 is positioned next-adjacent to the first protrusion 56. A depression 60 of a circular shape is disposed in the first protrusion 56 and a projection 62 of an oval shape extends upwardly from the first protrusion 56.

During a medical procedure, a user will position the second opening 44 of the adapter 20 with an end of the trocar 26. The user will then slide the first end of the trocar 26 into the second opening 44 of the adapter 20 to attach the adapter 20 to the trocar 26. Once attached, the flange 28 of the trocar 26 will be positioned in the recess 50 of the adapter 20. With the adapter 20 attached to the trocar 26, the user may position the second end of the trocar 26 adjacent a surgical site where a biopsy is to be obtained. Next, the user will position the cannula 22 and the stylet 24 above the adapter 20 and then lower the cannula 22 and the stylet 24 transversely to the lower surface 38 of the adapter 20. Once the cannula 22 and the stylet 24 are positioned on the lower surface 38, the user will slide the cannula 22 and the stylet 24 towards the first opening 42 in the groove 52 and the pair of rails 46 and the groove 52 will guide the cannula 22 and the stylet 24 into the first opening 42 and into the trocar 26. With the cannula 22 and the stylet 24 in the trocar 26, the user may extend the cannula 22 and the stylet 24 outwardly from the second end of the trocar 26 to obtain a biopsy of the surgical site.

Obviously, many modifications and variations of the present invention are possible in light of the above teachings and may be practiced otherwise than as specifically described while within the scope of the appended claims. That which is prior art in the claims precedes the novelty set forth in the “characterized by” clause. The novelty is meant to be particularly and distinctly recited in the “characterized by” clause whereas the antecedent recitations merely set forth the old and well-known combination in which the invention resides. These antecedent recitations should be interpreted to cover any combination in which the inventive novelty exercises its utility. The use of the word “said” in the apparatus claims refers to an antecedent that is a positive recitation meant to be included in the coverage of the claims whereas the word “the” precedes a word not meant to be included in the coverage of the claims. In addition, the reference numerals in the claims are merely for convenience and are not to be read in any way as limiting.

What is claimed is:

1. An adapter (20) for guiding a cannula (22) and a stylet (24) into a passageway of a trocar (26) having a flange (28), comprising;
said adapter (20) having a long end (30) and a short end (32) with said short end (32) being spaced from said long end (30) and a pair of edges (34) extending between and converging to connect to said long end (30) and said short end (32),
said adapter (20) having an upper surface (36) extending between said ends (30, 32) and said pair of edges (34),
said adapter (20) having a lower surface (38) extending between said ends (30, 32) and said pair of edges (34),
said adapter (20) defining a channel (40) extending between said ends (30, 32),
said adapter (20) defining a first opening (42),
said short end (32) defining a second opening (44) for receiving the trocar (26),
and characterized by,
a pair of rails (46) extending upwardly from said lower surface (38) and longitudinally in a direction from said long end (30) toward said short end (32) to define a third opening between said rails above said lower surface (38) whereby the cannula (22) and the stylet (24) may be received transversely to said lower surface (38) and guided by said pair of rails (46) into said first opening (42).

2. An adapter (20) as set forth in claim 1 wherein said lower surface (38) defines a groove (52) perpendicular to said long end (30) for guiding the cannula (22) and the stylet (24) into said first opening (42).

3. An adapter (20) as set forth in claim 2 wherein said lower surface (38) defines a recess (50) being disposed adjacent said short end (32) for receiving the flange (28) of the trocar (26).

4. An adapter (20) as set forth in claim 3 further including an awning (48) positioned longitudinally between said ends (30, 32) and extending between and connecting said pair of rails (46) to define said first opening (42).

5. An adapter (20) as set forth in claim 4 further including an indicia (54) extending from said upper surface (36) next-adjacent to said short end (32) for identifying said second end of said adapter (20) for receiving the trocar (26).

6. An adapter (20) as set forth in claim 5 wherein said indicia (54) includes a first protrusion (56) of a rectangular shape extending from said upper surface (36) and a second protrusion (58) extending from said upper surface (36) and having the shape of an arrow pointing to said long end (30) and being next-adjacent to said first protrusion (56).

7. An adapter (20) as set forth in claim 6 wherein said indicia (54) includes a depression (60) of a circular shape disposed in said first protrusion (56).

8. An adapter (20) as set forth in claim 7 wherein said indicia (54) includes a projection (62) of an oval shape extending upwardly from said first protrusion (56).

9. An adapter (20) for guiding a cannula (22) and a stylet (24) into a passageway of a trocar (26) having a flange (28), comprising;
said adapter (20) being of a trapezoidal shape including a long end (30) and a short end (32) with said short end (32) being spaced from said long end (30) and a pair of edges (34) being beveled inwardly and extending in an arcuate shape between and converging to connect to said long end (30) and said short end (32) to render said long end (30) longer in length than said short end (32),
said adapter (20) having an upper surface (36) extending between said ends (30, 32) and said pair of edges (34),
said adapter (20) having a lower surface (38) extending between said ends (30, 32) and said pair of edges (34),
said adapter (20) defining a channel (40) having a funnel shape extending between said ends (30, 32),
said adapter (20) defining a first opening (42),
said short end (32) defining a second opening (44) for receiving the trocar (26),
and characterized by,
a pair of rails (46) extending upwardly from said lower surface (38) and longitudinally in a direction from said long end (30) toward said short end (32) and converging to be parallel with said pair of edges (34) to define a third opening between said rails above said lower surface (38) whereby the cannula (22) and the stylet (24) may be received transversely to said lower surface (38) and guided by said pair of rails (46) into said first opening (42),
an awning (48) positioned longitudinally between said first end and said second end and extending between and connecting said pair of rails (46) to define said first opening (42),
said lower surface (38) of said adapter (20) defining a recess (50) of an oval shape being disposed adjacent said short end (32) and extending from said second opening (44) into said channel (40) for receiving the flange (28) of the trocar (26),
said lower surface (38) of said adapter (20) defining a groove (52) of a V-shape perpendicular to said long end (30) and extending from said first opening (42) to said recess (50) for guiding the cannula (22) and the stylet (24) into said first opening (42),
an indicia (54) extending from said upper surface (36) next-adjacent to said short end (32) for identifying said second end of said adapter (20) for receiving the trocar (26),
said indicia (54) including a first protrusion (56) of a rectangular shape extending from said upper surface (36) and a second protrusion (58) extending from said upper surface (36) and having the shape of an arrow pointing to said long end (30) and being next-adjacent to said first protrusion (56),
a depression (60) of a circular shape disposed in said first protrusion (56),
a projection (62) of an oval shape extending upwardly from said first protrusion (56).
